(19) United States
(12) Patent Application Publication
(10) Pub. No.: US 2007/0142424 A1
(43) Pub. Date: Jun. 21, 2007

(54) Process for producing tacrolimus (FK-506) using vegetable oil as sole source of carbon

(76) Inventor: Sudhir Vaid, Sterling City (IN)

Correspondence Address: BLANK ROME LLP, 600 NEW HAMPSHIRE AVENUE, N.W., WASHINGTON, DC, 20037, US

(21) Appl. No.: 11/698,881

(22) Filed: Jan. 29, 2007

(30) Foreign Application Priority Data
Jul. 29, 2004 (IN) 801/MUM/2004

(51) Int. Cl. A61K 31/4745; C12P 17/00; C12N 1/20

(52) U.S. Cl. 514/291; 435/117; 435/252.35

(57) ABSTRACT

A novel fermentation fed batch process for producing the immunosuppressant Tacrolimus (FK-506), using the mutant microorganism Streptomyces sp. utilizing vegetative oil as sole source of carbon in production medium.

### CROSS REFERENCE TO RELATED APPLICATIONS

This application is related to, and claims priority to, PCT Patent Application No. PCT/IN2005/000120, entitled “Novel Process For Producing Tacrolimus (FK-506) Using Vegetable Oil As Sole Source of Carbon,” filed on Apr. 19, 2005, and Indian Patent Application No. 801/MUM/2004, entitled “Novel Process For Producing Tacrolimus (FK-506) Using Vegetable Oil As Sole Source of Carbon,” filed on Jul. 29, 2004, which are expressly incorporated by reference herein.

### BACKGROUND OF THE INVENTION

1. Field of the Invention

This invention describes a novel process for producing tricyclo compounds such as FK-506 having pharmacological activities and to a pharmaceutical composition containing the same, wherein the sole source of carbon in the said process is vegetative oil.

The invention further provides a mutant strain, process for production of the tricyclo compounds by fermentation and then isolation of same by chemical means.

2. Description of the Related Art

Cyclosporin A, is an extremely effective anti rejection drug that revolutionized the field of organ transplant surgery e.g. bone marrow and heart transplants. Also, it is useful in graft versus host diseases and the like. The drug acts by inhibiting the body's immune system from mobilizing its vast arsenal of natural protecting agents to reject the transplant's foreign protein.

However, the effectiveness of the drug in fighting transplantation rejection is limited because it suffers drawbacks in causing kidney failure, liver damage, and ulcers, which in many cases can be severe.

U.S. Pat. No. 5,496,727 describes a process of fermentation of a particular strain of Streptomyces tsukubaensis—utilizing glucose, glycerin, starch dextrin, xylose and galactose as source of carbon to produce Tacrolimus FK-506 a potent immunosuppressant. Further, it also describes the closely related macrolide immunosuppressant FK-520 (FR-900520), produced by S. hygroscopicus sp. yakushimaensis. It is also exemplified that the source of carbon in both seed and production medium is sugar.

U.S. Pat. No. 5,352,783 describes a process for microbial transformation of a macrolide immunosuppressant by microorganism Streptomyces sp. utilizing the sugars viz. dextrose and dextrin as source of carbon in seed medium and in production medium also the source of carbon used is sugar viz. dextrose.

U.S. Pat. No. 5,110,811, U.S. Pat. No. 5,116,756, U.S. Pat. No. 5,225,403 and U.S. Pat, No. 5,225,403 describe a process for the production of FK-506 by fermentation of a particular strain of Streptomyces tsukubaensis utilizing glucose, glycerin, starch, dextrin, xylose and galactose as source of carbon. It is also exemplified that the source of carbon in both seed and production medium is sugar. Also, the growth of the strain on agar is very slow.

Also, U.S. Pat No. 4,956,352, claimed a process for the production of FK506 by fermentation of a particular strain of Streptomyces tsukubaensis no. 9993 under deposit No. FERM BP 927 utilizing glucose, Sucrose, Lactose, glycerin, starch, dextrin, and the like as source of carbon. Though, the strain is Streptomyces tsukubaensis no. 9993, the source of carbon in both seed and production medium is sugar.

However, the activity obtained in the fermentation broth is very less, as per the processes mentioned in the literature. Hence, there is a need to improve the activity obtained in the fermentation broth. Also, the source of carbon in both seed and production medium is sugar. The present invention describes a process of producing tricyclo compounds Tacrolimus by fermentation of mutant microorganisms, utilizing alternate sole source of carbon such as vegetative oil, are highly desired in the field.

### SUMMARY AND OBJECTS OF THE INVENTION

The immunosuppressant Tacrolimus FK506 is directly obtained by fermentation. The process is preferably conducted under submerged aerobic conditions using a mutant strain wherein the sole source of carbon is vegetative oil in the production medium.

This invention describes a novel process for producing tricyclo compounds having pharmacological activities and to a pharmaceutical composition containing the same, wherein the sole source of carbon in the said process is vegetative oil.

An objective of this invention is to provide a process for production of the tricyclo compounds by fermentation and then isolation of same by chemical means.

With respect to present invention it is to be noted that this invention is originated from and based on first and new discovery of compounds like FR-900506, FR-900520, and compounds like FR-900506. These compounds were produced in culture broths obtained by fermentation of species related to genus streptomyces.

### DETAILED DESCRIPTION OF THE INVENTION

Although preferred embodiments of the invention are described for illustrative purposes, it should be understood that the invention may be embodied in other forms not specifically described or exemplified.

The present invention describes a process which involves mutation of strains belonging to genus streptomyces by conventional means such as UV radiation, X-ray radiation and the like and then culturing of the same in a nutrient media using vegetative oil as a sole carbon source to produce novel tricyclo compounds like FK-506, FK-520 and the like:

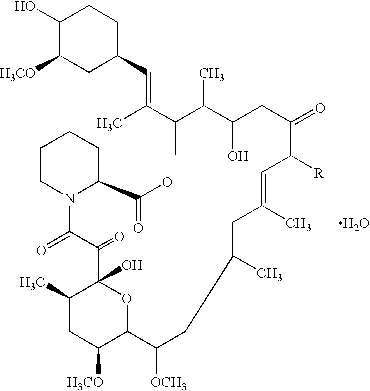

Where R→CH₂CH═CH₂ (FK-506)

Where R→CH₂—CH₂—CH₃ (FK-520)

According to the present invention the fermentation can be done of any known species, such as Streptomyces sp. FERM BP 0927, Streptomyces sp., (MA 6858) ATCC No. 55098 etc., which produces FK 506. The physical characteristics and taxonomy, including morphological, cultural, biological and physiological characteristics are briefly described herein below.

The Streptomyces sp. strain P5C3, FERM BP 0927 grows very slowly on agar media and a preferred growth medium is Yeast Malt Extract Agar at 28 degrees Celsius. The present invention process can be practiced with any FK-506 producing strain of Streptomyces sp., and particularly preferred is the herein described mutant P5C3.

In general, FK-506 can be produced by culturing (fermenting) the various strains known in the literature, including the above described mutant strain in aqueous nutrient medium, containing vegetative oil as sole source of assimilable carbon, and nitrogen, preferably under submerged aerobic conditions (e.g. shaking culture, submerged culture, etc). The aqueous medium is preferably maintained at a pH of about 6.0-8.5 at the initiation and termination (harvest) of the fermentation process.

The sole sources of carbon in the nutrient medium are vegetable oils such as cotton seed oil, groundnut oil, soya oil, sunflower oil, vegetable oil, and the like. The preferred sources of nitrogen are yeast extract, meat extract, peptone, gluten meal, cottonseed meal, soybean meal and other, vegetable meals (partially or totally defatted), casein hydrolysates, soybean hydrolysates and yeast hydrolysates, corn steep liquor, dried yeast, wheat germ, feather meal, peanut powder, distiller's solubles, etc., as well as inorganic salts (e.g. ammonium nitrate, ammonium-sulfate, ammonium phosphate, etc.,), urea, amino acids, and the like.

The carbon and nitrogen sources, though advantageously employed in combination, need not be used in their pure form, because less pure materials which contain traces of growth factors and considerable quantities of mineral nutrients, are also suitable for use. When desired, these may be added to the medium mineral salts such as sodium or calcium carbonate, sodium or potassium phosphate, sodium or potassium chloride, sodium or potassium iodide, magnesium salts, copper salts, cobalt salts, and the like. If necessary, especially when the culture medium foams seriously, a defoaming agent, such as liquid paraffin, fatty oil, plant oil, mineral oil or silicone may be added.

As to the conditions for production of FK-506 in massive amounts, submerged aerobic cultural conditions are preferred therefore. For the production of small amounts, a shaking or surface culture in a flask, bottle or culture dish is employed. Furthermore, when the growth is carried out in large tanks, it is preferable to use the vegetative form of the organism for inoculation in the production tanks in order to avoid growth lag in the process of production of FK-506. Accordingly, it is desirable first to produce a vegetative inoculum of the organism by inoculating a relatively small quantity of culture medium with spores or mycelia of the organism produced in a “slant’ and culturing said inoculated medium, also called the “seed medium”, and then to transfer the cultured vegetative inoculum aseptically to large tanks. The fermentation medium, in which the inoculum is produced, is substantially the same as or different from the medium utilized for the production of FK-506 and is generally autoclaved to sterilize the medium prior to inoculation. The pH of the medium is generally adjusted to about 7.0 prior to the autoclaving step by suitable addition of an acid or base, preferably in the form of a buffering solution.

Agitation and aeration of the culture mixture may be accomplished in a variety of ways. Agitation may be provided by a propeller or similar mechanical agitation equipment, by revolving or shaking the fermenter, by various pumping equipment or by the passage of sterile air through the fermentation mixture. The fermentation is usually conducted at a temperature between about 20° C. and 40° C., preferably between 24° C. and 30° C., for a period of 48 hours 216 hours, which may be varied according to fermentation conditions and scales. Preferably, the production cultures are incubated for about 216 hours at 25° C. on a rotary shaker operating at 240 rpm, wherein the pH of the fermentation medium is maintained at 7.0 by continuous feeding of vegetable oil till harvest.

The fermentation is carried out at pH 6.0 to 8.5 for up to 300 hrs in case of FK-506. Preferably, the fermentation is carried out for 120-268 hrs.

Preferred culturing/production media for carrying out the fermentation include the following:

Seed Medium A

| Ingredient | g/L |
|---|---|
| Corn Steep Liquor | 25.0 |
| Sucrose | 18.0 |
| Ammonium Sulphate | 1.5 |
| Calcium Carbonate | 7.2 |
| Soya Oil | 8.0 |

Adjust pH to 7.0

Production Medium B

| Ingredient | g/L |
|---|---|
| Ammonium Sulfate | 0.50 |
| Calcium Carbonate | 4.50 |
| Pea Nut Meal | 15.0 |
| Yeast Extract | 5.0 |
| Soya Bean Flour | 3.5 |
| Soya Oil | 35.0-75 |

Adjust pH to 6.8.

The produced FK-506 can be recovered from the culture medium by extraction in toluene. The toluene extract then concentrated, column chromatographed over silica gel using ethyl acetate:hexane mixture to get crude Tacrolimus. The crude substance further purified on preparative HPLC to get Tacrolimus pure.

Thus, the described process is for producing the immunosuppressant Tacrolimus (FK-506), using the mutant strains selected from the group comprising of streptomyces sp. Strain P5C3, Streptomyces sp. FERM BP 0927, Streptomyces sp., (MA 6858) ATCC No. 55098. The said process comprises of culturing a mutant strain of Streptomyces sp. under submerged aerobic fermentation conditions in an aqueous nutrient medium containing vegetable oil as a sole source of carbon along with a nitrogen source. The source of oil is preferably selected from cotton seed oil, ground nut oil, soya oil, sunflower oil, and vegetable oil. The source of oil is preferably soya oil. According to the process 6.0 to 8.5 pH is maintained throughout the process till harvest by continuous feeding of vegetable oil. The time required for the process is up to 300 hrs. The time required for the process is preferably 120 to 268 hrs. A batch fed process according to the instant invention using a mutant strain having a continuous feeding of vegetable oil as assimialable sole source of carbon.

The following examples are given for the purpose of illustrating the present invention and should not be construed as being limitations on. the scope or spirit of the instant invention.

### EXAMPLE 1

Mutagenesis and Culture Conditions

Spore suspension in Normal Saline (0.85%) of P5C3 are prepared from YM agar (Yeast extract, 0.4%, malt extract, 1.0%, glucose 0.4%, pH adjusted to 7.0. and agar, 2.0%) and are exposed to ultra-violet radiations. The treated spores are plated for isolated colonies on YM agar. The subject mutant, P5C3, was identified as an isolate which produces a major component utilizing vegetative oil as sole carbon source. Estimation of the FK-506 produced by the mutant phenotype is through fermentation, extraction and then HPLC analyses of reisolates of the original colony “patch”. The fermentation involves inoculating a 250 ml Erlenmeyer flask containing 50 ml of an autoclaved seed medium A prepared with distilled water. The seed medium is inoculated with spores from YM agar medium and incubated for 42-48 hours at 26-32° C. on a rotary shaker operating at 240 rpm. After 48 hours of growing in seed medium a PMV of 12-18% is achieved with a pH of 7-8.5 with morphological characteristics like long thin branched mycelia forming a closely knit network in the shape of clumps. A 1.0 ml aliquot of the resulting seed culture is used to inoculate a 250 ml Erlenmeyer flask containing production medium B prepared with distilled water, where the pH is adjusted to 6.8 with NaOH prior to autoclaving. The production culture is incubated for 216 hours, at 26-32° C. on a rotary shaker operating at 240 rpm. An acetonitrile extraction is achieved by addition of a double volume of acetonitrile to the broth culture, agitating at high speed on wrist shaker for 30 minutes. The aqueous acetonitrile extracts are analyzed by HPLC. The titer of tacrolimus (FK-506) by this process ranges between 150-250 mg/L.

### EXAMPLE 2

50 ml of a medium for seed culture (pH 6.5) containing glycerin 1%, soluble starch 1%, glucose 0.5%, cotton seed meal 0.5%, dried yeast powder 0.5%, corn steep liquor 0.5%, and calcium carbonate 0.2% was added to 500 ml Erlenmeyer flask, and autoclaved at 121° C. for 30 min. 0.05 ml of slant culture of Streptomyces tsukubaensis grown for 9-11 days, which was harvested with 3.0 ml of normal saline and incubated under shaking of reciprocatory shaker with 240 rpm and 5 cm displacement, at 28° C. for 48-72 hrs. Then 5.0 ml of mature seed with pH in the range of 6.0-8.0 and with pmv 10-30% is transferred to 50 ml of production media containing dextrose 2.5%, dextrin white 8%, glycerol 10%, cotton seed meal 1%, soya bean meal 1%, PEG 400 0.1%, soya peptone 1%, KH₂PO₄ 0.08%, CaCO₃ (L) 0.15% and final pH adjust to 7.0. The inoculated production flask was incubated at 24° C.-28° C. for 10 days under shaking at 240 rpm on a reciprocatory shaker with 5 cm displacement/throw.

Tacrolimus was quantitatively assayed by HPLC. After 10 days of incubation pH reached to 6.8-7.8, pmv to 30-40% and fermentation titre 30-60 mg/l.

### EXAMPLE 3

50 ml of a medium for seed culture (pH 7.0) containing corn steep liquor 2.5%, sucrose 1.8%, (NH₄)₂SO₄ 0.15%, CaCO₃ 0.72%, soya oil 0.72% w/v, was added to 500 ml Erlenmeyer flask and autoclaved at 121° C. for 30 min. 0.05 ml of slant culture of Streptomyces tsukubaensis grown for 9-11 days which was harvested with 3.0 ml of normal saline and incubated under shaking at rotary shaker with 240 rpm and 5 cm displacement at 28° C. for 48-72 hrs.

Then 5.0 ml of mature seed with pH in the range of 7.0-8.5 and with pmv 10-30% is transferred to 50 ml of production media containing (NH₄)₂S0₄ 0.03%, CaCO₃ 0.337%, ground nut meal 1.64%, soya bean meal 0.5%, yeast extract 0.49%, soya oil 4.0% and the final pH adjust to 7.2 with 40% NaOH. The said seeded production flask was incubated at 28° C. for 10 days under shaking at 240 rpm on a rotary shaker with 5 cm displacement/throw.

Tacrolimus was quantitatively assayed by HPLC. After 10 days of incubation pH reached to 7.0-8.5, pmv to 40-55% and titre 90-120 mg/L.

### EXAMPLE 4

Without Fed Batch:

600 L of a medium for seed culture (pH 7.0-7.2) containing 18 kg sucrose, corn steep liquor 18 kg, (NH₄)₂S0₄ 1.2 kg, CaCO₃ 3.9 kg, soya oil 4.8 kg, antifoam 30 ml, sterilized at 121° C. for 60 min. and inoculated with 1 liter spores of Streptomyces tsukubaensis grown for 9-11 days in 1 Liter flask containing 100 ml of potato glycerol agar incubated at 28° C., which was harvested with 1 liter of saline.

Then 600 L of matured seed with pH in the range of 7.0-8.5, pmv 10-20% is transferred to 6000 L of production media containing ground nut cake 98.4 kg, soya bean meal 30 Kg, (NH₄)₂S0₄ 1.8 kg, yeast extract 29.6 kg, CaCO₃ 20.2 kg, soya oil 240 L, Antifoam 300 ml and the final pH adjusted into 7.2 with 40% NaOH. The said seed medium was transferred to production media in a ratio of 10% v/v and incubated at 28° C. for 4-6 days.

Tacrolimus was quantitatively assayed by HPLC. After 6-8 days of incubation pH reached to 7-8.5, packed mycelial volume to 25-35% and titre 127-180 mg/L.

### EXAMPLE 5

Fed Batch:

600 L of a medium for seed culture (pH 7.0-7.2) containing 18 kg sucrose, corn steep liquor 18 kg, (NH₄)₂S0₄ 1.2 kg, CaCO₃ 3.9 kg, Soya oil 4.8 kg, antifoam 30 ml, sterilized at 121° C. for 60 min. and inoculated with 1 liter spores of Streptomyces tsukubaensis grown for 9-11 days in, 1 liter flask containing 100 ml of potato glycerol agar incubated at 28° C., which was harvested with 1 liter of saline. Then 600 L of matured seed with pH in the range of 7.0-8.5, pmv 10-20% is transferred to 6000 L of production media containing ground nut cake 98.4 kg, soya bean meal 30 kg, (NH₄)₂S0₄ 1.8 kg, yeast extract 29.6 kg, CaCO₃ 20.2 kg, soya oil 240 L, antifoam 300 ml and the final pH adjusted into 7.2 with 40% NaOH. The said seed medium was transferred to production medium in a ratio of 10% w/v and incubated at 25-26° C. for 4-6 days. The Soya oil is used as a feed after 48 hours to maintain the pH at 7.0-7.2 and the oil level is maintained between 0.2-0.7%.

Tacrolimus was quantitatively assayed by HPLC. After 6-8 days of incubation pH reached to 7-8.5, packed cell mycelial volume (pmv) to 45-55% and titre 180-330 mg/L.

It is reasonably believed that a mutation in the organism has made it capable of utilizing a higher concentration of vegetative oil and used as sole carbon source other than carbohydrates for the production of Tacrolimus (FK-506). Feeding of soya oil in batch fed helped to maintain the pH hence improved packed cell mycelial volume (pmv) and fermentation titer in shorter incubation time.

We claim:

1. A process for producing a tricyclo compound including tacrolimus comprising the step of:
(a) providing a strain selected from the group consisting of Streptomyces sp. Strain P5C3 and Streptomyces sp. (MA 6858) ATCC No. 55098.

2. The process of claim 1, further comprising the step of:
(b) culturing the strain using aerobic fermentation in an aqueous nutrient medium, wherein a vegetable oil is the sole source of carbon in the medium.

3. The process of claim 2, wherein the vegetable oil is selected from the group consisting of cotton seed oil, ground nut oil, soya oil, sunflower oil, and mixtures thereof.

4. The process of claim 2, wherein the vegetable oil comprises soya oil.

5. The process of claim 4, wherein the soya oil is continuously fed in a batch fed process.

6. The process of claim 2, wherein the medium is maintained at a pH between about 6.0 and 8.5.

7. The process of claim 2, wherein the aerobic fermentation comprises submerged aerating the culture medium.

8. The process of claim 2, wherein the aerobic fermentation comprises agitating the culture medium.

9. The process of claim 2, wherein the aerobic fermentation is conducted at a temperature between about 20° C. and 40° C.

10. The process of claim 2, wherein the aerobic fermentation is conducted for a time period between about 48 hours and 300 hours.

11. The process of claim 2, wherein the aerobic fermentation is conducted for a time period sufficient to substantially inhibit the production of tacrolimus.

12. The process of claim 2, further comprising the step of:
(c) isolating the cultured tricyclo compound.

13. The process of claim 12, wherein the step of isolating comprises adding an organic solvent to the medium to extract the tricyclo compound, separating the extract from the medium, and removing the solvent from the extract.

14. The process of claim 12, further comprising the step of:
(d) selecting a pharmacologically active amount of the isolated tricyclo compound for use in a pharmaceutical composition.

15. The process of claim 2, wherein the strain has been mutated before culturing.

16. A pharmaceutical composition comprising tacrolimus produced from a strain selected from the group consisting of Streptomyces sp. Strain P5C3 and Streptomyces sp. (MA 6858) ATCC No. 55098.

17. The pharmaceutical composition of claim 16, wherein the tacrolimus is produced by culturing the strain of the Streptomyces sp. using aerobic fermentation in an aqueous nutrient medium, and then isolating the cultured tacrolimus, wherein a vegetable oil is the sole source of carbon in the medium.

18. The pharmaceutical composition of claim 16, wherein the tacrolimus is present in a pharmacologically active amount.

19. A process for producing tacrolimus by culturing a mutated strain of Streptomyces in a growth medium using submerged aerobic fermentation and then isolating the culture, wherein a vegetative oil is the sole source of carbon in the medium.

20. The process of claim 19, wherein the strain is a strain of the species Streptomyces tsukubaensis.

21. The process of claim 19, wherein the strain is P5C3.

22. The process of claim 19, wherein the vegetable oil is selected from the group consisting of cotton seed oil, ground nut oil, soya oil, sunflower oil, and mixtures thereof.

23. The process of claim 19, wherein the submerged aerobic fermentation is conducted at a temperature between about 20° C. and 40° C., and at a pH between about 6.0 and 8.5.

24. The process of claim 19, further comprising the steps of isolating the tacrolimus and then selecting a pharmacologically active amount of the isolated tacrolimus for use in a pharmaceutical composition.

25. A mutant strain of Streptomyces sp. Strain P5C3.
